SEQUENCE LISTING (2 sequences)

SEQ ID NO: 1 - DNA, 20 bp, artificial
  BCRP PCR forward primer
agtccggaaaacagctgaga

SEQ ID NO: 2 - DNA, 20 bp, rat
  BCRP reverse primer
cccatcacaacgtcatcttg

(19) United States
(12) Patent Application Publication
(10) Pub. No.: US 2006/0233809 A1
(43) Pub. Date: Oct. 19, 2006

(54) Method for treating prostate conditions

(76) Inventors: Gary J. Smith, Buffalo, NY (US); Wendy J. Huss, Kenmore, NY (US)

Correspondence Address: HODGSON RUSS LLP, ONE M & T PLAZA, SUITE 2000, BUFFALO, NY, 14203-2391, US

(21) Appl. No.: 11/350,171

(22) Filed: Feb. 8, 2006

Related U.S. Application Data
(60) Provisional application No. 60/651,101, filed on Feb. 8, 2005.

(51) Int. Cl. A61K 39/395; A61K 36/889; A61K 31/496; A61K 31/56; A61K 31/4745; A61K 31/137

(52) U.S. Cl. 424/155.1; 424/727; 514/283; 514/171; 514/254.09; 514/651

(57) ABSTRACT

The invention provides a method for inhibiting the aberrant growth of cells in a prostate tissue in an individual comprising administering to the individual an amount of an inhibitor of the Breast Cancer Resistance Protein (BCRP/ABCG2), where the amount of the BCRP inhibitor is effective to inhibit the growth of the aberrantly growing cells. The method is also useful for treating prostate tumors or benign prostatic hyperplasia/hypertrophy (BPH). Also disclosed is the phenotype for prostate stem cells as determined by immunohistochecmical localization methods. The prostate stem cells are positive for BCRP protein, negative for androgen receptor protein, negative for p63 protein, and negative for synaptophysin.

This application claims priority to U.S. patent application Ser. No. 60/651,101, filed on Feb. 8, 2005, the disclosure of which is incorporated herein in its entirety.

This work was supported by Grant No. CA 77739 from the National Cancer Institute. The Government has certain rights in the invention.

### FIELD OF THE INVENTION

The present invention relates generally to treatment of prostate cancer and more specifically to compositions and methods for inhibiting the aberrant growth of prostate cells in prostate tumors and benign prostatic hyperplasia.

### BACKGROUND OF THE INVENTION

Prostate cancer is the most common type of cancer in men in the U.S. and the second leading cause of cancer related death. In its advanced stages, prostate cancer metastasizes, among other tissues, to bone. Prostate cancer is often treated by androgen deprivation therapy. However, after initial response, most metastatic prostate cancers become hormone-refractory and eventually lethal. Current therapeutic approaches have not been able to treat androgen deprivation resistant tumors.

Although the reasons for emergence of androgen deprivation resistance are not clearly understood, it appears that treatment regimes designed to reduce the bulk of a tumor or eliminate the proliferative compartment do not target the phenotypically distinct stem cells (Reya et al., Nature (2001); 414:105-11). The stem cells are androgen deprivation resistant and therefore continue to provide a source of proliferative cells after androgen deprivation therapy.

Resistance to other agents, such as chemotherapeutic agents, is sometimes associated with the presence of cell membrane pumps of the ABC family (such as the multi-drug resistance protein) that are able to effectively transport the agent out of the cell. Specifically, Breast Cancer Resistance Protein (“BCRP” or ABCG2) has been implicated in transport of chemotherapeutic agents out of tumor cells (Doyle et al., Oncogene (2003); 22:7340-58). However, it is not known if androgen deprivation resistance is in any way related to the presence of a similar pump in prostate cells. Therefore, there is an ongoing need for understanding the reasons for androgen deprivation resistance and consequently for identifying novel prostate cancer therapies which could target putative prostate stem cells.

### SUMMARY OF THE INVENTION

The present invention is based upon the discovery of cells in prostate tissue which express BCRP protein, and which lack androgen receptor protein (“AR”), p63 protein, and synaptophysin, as determined by immunohistochemical localization. BCRP is demonstrated herein to mediate androgen efflux from these cells. It is believed that BCRP mediated androgen efflux causes these cells to be resistant to conventional prostate cancer therapies and to serve as a nidus of aberrantly growing cells. Thus, the present invention provides a method for reducing the aberrant growth of cells in a prostate tissue comprising the step of administering to an individual a therapeutically effective amount of a composition comprising a BCRP inhibitor, wherein administration of the composition comprising the BCRP inhibitor reduces the aberrant growth of the prostate tissue cells.

Any BCRP inhibitor suitable for administration to humans can be used in the method. In one embodiment, novobiocin is used as the BCRP inhibitor. The BCRP inhibitor can be administered concurrently or sequentially with conventional prostate cancer therapies, such as chemotherapies and/or radiation therapy. Further, while androgen deprivation therapy is routine in the treatment of prostate tumors, the method can be used independent of androgen deprivation therapy, or in combination with continuous or intermittent androgen deprivation. Additionally, the method is useful for treating benign prostatic hyperplasia/hypertrophy (BPH).

### BRIEF DESCRIPTION OF THE FIGURES

The patent or application file contains at least one drawing executed in color. Copies of this patent or patent application publication with the color drawings will be provided by the Office upon request and payment of the necessary fee.

FIGS. 1A through FIG. 1K are photographic representations of immunohistochemical staining in putative human prostate stem cells. FIGS. 1A to 1E represent benign human prostate tissue immunostained for (FIG. 1A) BCRP (blue) and AR (red); (FIG. 1B) BCRP (blue) and p63 (red); (FIG. 1C) BCRP (blue) and high molecular weight cytokeratin (red); (FIG. 1D) BCRP (blue) and synaptophysin (red); (FIG. 1E) BCRP (blue) and smooth muscle α-actin (red). FIG. 1F represents human prostate cancer immunostained for BCRP (blue) and AR (red). FIGS. 1G to 1I represent human prostate xenografts harvested after 30 days of androgen deprivation and 2 days of dihydrotestosterone stimulation immunostained for (FIG. 1G) BCRP (blue) and AR (red); (FIG. 1H) BCRP (blue) and p63 (red); (FIG. 1I) BCRP (blue) and Ki67 (red). FIGS. 1J and 1K represent needle biopsies harvested from an advanced prostate cancer patient undergoing hormonal therapy immunostained for BCRP (blue) and AR (red) before initiation of therapy (FIG. 1J) and 1 month after initiation of hormonal therapy (FIG. 1K). Black arrows, BCRP+/AR−/p63− cells; Black arrowheads, p63 (B), high molecular weight cytokeratin (C), synaptophysin (D), smooth muscle α-actin (E). Red arrows, foci of BCRP+ cells. Green arrows, proliferating, Ki67-expressing cells. Bar, 20 μm.

FIGS. 2A through FIG. 2K are photographic representations of immunohistochemical staining in prostate stem cells during the emergence of neuroendocrine-like carcinomas in the TRAMP model (Transgenic Adenocarcinoma of the Prostate). FIGS. 2A to 2C represent serial sections of ventral prostate from a TRAMP animal 4 days postcastration immunostained for (FIG. 2A) AR (brown), (FIG. 2B) SV40Tag (brown), and (FIG. 2C) Ki67 (blue) and synaptophysin (pink). FIGS. 2D to 2F represent serial sections of ventral prostate from a TRAMP animal 14 days postcastration immunostained for (FIG. 2D) Foxa2 (brown), (FIG. 2E) SV40Tag (brown), and (FIG. 2F) BCRP (blue) and AR (red). FIG. 2G represents ventral prostate from a TRAMP animal 1 day postcastration immunostained for BCRP (blue) and AR (red). FIGS. 2H and 2I represent serial sections of ventral prostate harvested 14 days postcastration from animals pulsed with BrdUrd for 2 weeks before castration, immunostained for (FIG. 2H) BCRP (blue) and AR (red), and (FIG. 2I) BrdUrd (blue) and synaptophysin (red). FIGS. 2J and 2K represent serial sections of neuroendocrine-like carcinoma in prostates harvested 14 days postcastration immunostained for (FIG. 2J) BCRP (blue) and AR (red), and (FIG. 2K) Ki67 (blue) and synaptophysin (pink). Black arrowheads, AR−/SV40+/Syn+/Ki67+/Foxa2+ foci (transit/amplifying populations) postcastration. Black arrows, BCRP+/AR− stem cells. Red arrows, BCRP+ foci. Red arrowheads, location of BCRP+ cells in the matched serial section (FIGS. D, E, I,. and K), Bar, 20 μm.

FIGS. 3A through FIG. 3C are photographic representation of three different assays of inhibition of BCRP-mediated transport on AR expression in the rat prostate progenitor cell line, RPE. FIG. 3A represents an electrophoretic separation of products from an RT-PCR analysis of BCRP (486 bp) and AR (234 bp) expression in ventral (V) prostate, colon, and small (S) intestine of Fischer 344 rats and the DP2, DP3, DP4, and RPE rat prostate cell lines. FIG. 3B represents an immunocytochemical analysis of AR protein expression in RPE cells cultured in the presence and absence of 50 μmol/L novobiocin (Nov) and/or 3 nmol/L dihydrotestosterone (DHT). Bar, 20 μm. FIG. 3C represents an immunoblot analysis of AR (110 kDa), BCRP (70 kDa), and actin (42 kDa) expression in RPE cells cultured in the presence and absence of 50 μmol/L novobiocin and/or 3 nmol/L dihydrotestosterone.

FIG. 4 is a graphical representation of BCRP-mediated efflux of Hoechst 33342 and androgen in the Mx-RPE cell line. Inhibition of efflux of Hoechst 33342 (black columns) or [⁻³H]dihydrotestosterone (gray columns) in Mx·RPE cells by 5 or 10 mmol/L novobiocin (Nov) or 10 μmol/L fuimitremorgin C (FTC) compared with Hoechst 33342 or [⁻³H]dihydrotestosterone efflux in the absence of novobiocin or fumitremorgin C. Data for Hoechst 33342 efflux represent two independent experiments (n>1,000 cells, *** P<0.0001). Data for [⁻³H]dihydrotestosterone efflux represent three independent experiments, each done in triplicate wells (^{*}P<0.01; **P<0.001).

### DESCRIPTION OF THE INVENTION

The present invention is based upon the discovery of cells in the prostate which express BCRP. The BCRP positive cells (designated herein as “BCRP+”) were generally observed to be lacking the AR protein (as determined by immunohistochemical localization; referred to herein as “AR−”). Further, these cells were also observed to be lacking (as determined by immunohistochemical localization) the p63 protein (referred to herein as “p63−”) and synaptophysin (referred to herein as “Syn−”). These BCRP+/AR−/p63−/Syn− cells are termed herein as “prostate stem cells.” Throughout the application, the terms “prostate stem cells” and “BCRP+/AR−/p63−/Syn−” are used interchangeably.

The BCRP+/AR−/p63−/Syn− phenotype is distinct from that of the quasi-differentiated cells that form the bulk of a prostate tumor that are the progeny of the undifferentiated stem cell. Further, because of this phenotypic distinction between the prostate stem cells and those forming the bulk of the prostate tumor, current clinical treatment regimes that are targeted at reducing the bulk of a tumor will not kill the undifferentiated prostate stem cells which are androgen deprivation resistant, nor will they prevent the recurrent prostate cancer that occurs in essentially all of patients treated by androgen deprivation.

The data presented herein demonstrates that BCRP mediates androgen efflux in the prostate stem cells. Data obtained from human prostate tissue xenografts, an animal model for prostate adenocarcinoma tumors, and a human patient strongly suggest that the prostate stem cells provide a nidus of cells that exhibit aberrant growth. It is believed that in the prostate stem cells, BCRP-mediated efflux of androgen prevents accumulation of intracellular androgen. As a result, AR is unable to bind to its ligand, and is therefore degraded via the ubiquitin/proteosomal pathway preventing the cells from proceeding along a differentiation pathway.

While not intending to be bound by any particular theory, it is believed that the method of the present invention inhibits the ability of prostate stem cells to continue to act as a nidus of aberrantly growing cells. The method involves inhibiting BCRP mediated efflux of androgen by the administration of BCRP inhibitors. It is believed that, because of this inhibition, AR can bind to its ligand, avoid degradation via the ubiquitin/proteosomal pathway, and drive gene expression which promotes ultimate differentiation. Thus, it is believed that inhibition of the androgen mediated efflux function of BCRP inhibits the growth of prostate tumors by a mechanism which either induces prostate stem cells to exit the stem cell compartment and ultimately differentiate, or prevents prostate stem cell proliferation, resulting in a lack of maintenance of this compartment, and the ultimate senescence of the prostate tumor. It is also possible that with increased intracellular androgen and AR, and the resulting proliferation and differentiation, the prostate stem cells may become sensitive to androgen deprivation induced apoptotic death, or senesce and undergo programmed cell death.

Accordingly, the present invention provides a method for reducing aberrant growth of prostate tissue cells comprising the step of administering to an individual a therapeutically effective amount of a composition comprising one or more BCRP inhibitors. The administration of the composition comprising the BCRP inhibitor(s) reduces the aberrant growth of prostate tissue cells. Data obtained from a well-accepted animal model for prostate cancer indicates that a BCRP inhibitor reduced the number of foci made up of poorly differentiated cells considered to originate from prostate stem cells.

Examples of suitable BCRP inhibitors for use in the method of the invention include GF120918 (Doyle et al., Oncogene. (2003) Vol. 47:7340-58); fumitremorgin (FTC); other fumitremorgin-type indolyl diketopiperazines (van Loevezijn et al., (2001) Bioorg. Med. Chem. Lett., Vol. 11, 29-32); the tetracyclic FTC analog Kol43 (Allen et al. (2002) Mol. Cancer Ther., Vol. 1, 417-425); novobiocin (Doyle et al. (1996) Proc. Am. Soc. Clin. Oncol., Vol. 15, 398); antibodies to BCRP; the ErbB1 tyrosine kinase inhibitor CI1033 (Erlichman et al., (2001) Cancer Res., Vol. 61, 739-748); the ErbB1 inhibitor Iressa (ZD1839) (Schuetz et al., (2002) Proc. Am. Assoc. Cancer Res., Vol. 43, 272 (abstract 1351)); the rauwolfia alkaloid reserpine (Zhou et al., (2001) Nat. Med., Vol. 7, 1028-1034); the pipecolinate derivatives VX-710 (biricodar, Incel) and VX-853 (Baer et al., (2002) Blood (Suppl.), Vol. 100, 67a (abstract 245)); estrogen agonists and anti estrogens (Sugimoto et al., (2003) Mol. Cancer Ther., Vol. 2, 105-112); diethylstilbesterol, estrone, tamoxifen, toremifene, and the tamoxifen derivatives TAG-11 and TAG-1 39 (Doyle et al., Oncogene (2003) Vol. 47:7340-58). A preferred BCRP inhibitor is novobiocin, which is well tolerated by humans.

The BCRP inhibitors can be formulated in pharmaceutically acceptable carriers which are well known to those skilled in the art. The pharmaceutical compositions can then be delivered by any suitable administration route, such as parenteral, e.g., intravenous, intradermal, subcutaneous, oral (e.g., inhalation), transdermal (topical), transmucosal, and rectal administration, or by direct injection into the prostate. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic. Further, the dosage and administration of BCRP inhibitors are well within the purview of those skilled in the art. In one embodiment, the BCRP inhibitor novobiocin is given daily by oral administration in a dosage of 4 gm/day in 250 mg tablets.

A composition comprising a BCRP inhibitor may be administered to an individual diagnosed with a prostate condition such as prostate tumor or BHP. In one embodiment, the BCRP inhibitor is administered to an individual undergoing androgen deprivation therapy. However, the present invention is also useful for treating individuals with prostate tumors, irrespective of whether the individuals are undergoing androgen deprivation therapy. When the method is practiced in conjunction with androgen deprivation, the andgrogen deprivation can be achieved in a variety of ways known to those skilled in the art. For example, the individual may undergo orchiectomy (castration) to inhibit endogenous androgen production by the testes. Additionally, various anti-androgenic hormone therapies are known in the art to inhibit androgen production. For example, luteinizing hormone-releasing hormone (LHRH) analogs, such as leuprolide, goserelin or triptorelin, or commercially available LHRH antagonists may be administered by conventional means. Further, antiandrogens, such as flutamide, bicalutamide, and nilutamide, or other androgen-suppressing drugs, such as estrogens, may be administered. The androgen deprivation may be intermittent or continuous, and may be initiated before, during or after administration of the BCRP inhibitors. In one embodiment, an exogenous androgen can be administered to an individual who is not undergoing androgen deprivation therapy, or who is undergoing an intermittent withdrawal of androgen deprivation therapy. Suitable androgens are known to those skilled in the art.

In another embodiment, the BCRP inhibitor can be administered with conventional chemotherapeutic agents and/or radiation therapy. Radiation may be delivered either via external beam radiotherapy or via local placement of radioactive seeds within the prostate (brachytherapy). In this regard, the BCRP inhibitor can be administered with conventional chemotherapeutic agents or radiation therapy concurrently or sequentially.

As will be recognized by those skilled in the art, the method of the invention will also be useful for inhibiting the aberrant enlargement of the prostate characteristic of BPH. When the method is used to treat an individual with BPH, administration of the BCRP inhibitor can be performed in combination with therapies utilizing commercially available alpha-blockers (such as Cardura® or doxazosin mesylate; Flomax® or tamsulosin hydrochloride; Hytrin® or terazosin hydrochloride; Minipress® or prazosin hydrochloride; and Uroxatral® or alfuzosin hydrochloride) or alpha-reductase inhibitors (such as Proscar® or finasteride; and Avodart® or dutasteride), or herbal remedies, such as saw palmetto.

The following Examples are meant to illustrate the invention and are not meant to be limiting.

### Example 1

This Example demonstrates the identification of prostate stem cells in human prostate tissue and primary xenografts.

All human prostate specimens referred to in this and other Examples herein were excess tissue harvested at the time of radical prostatectomy or needle biopsies harvested during androgen deprivation therapy, in accordance with NIH guidelines for use of human subjects, with approval by the Institutional Review Board at University of North Carolina.

All experiments using laboratory animals in this and other Examples herein were performed in accordance with Institutional Animal Care and Use Committee and NIH guidelines.

Cells in human prostate tissue can be analyzed using standard in vitro assays and by using animal models of prostate cancer. We have recently developed one such model using male athymic nude mice as hosts for human prostate tissue xenografts (Huss et al., Prostate (2004); 60:77-90). Such xenografts can be established from both freshly harvested and cryopreserved tissue fragments. Histology of xenografts of malignant and benign prostates are generally consistent with their respective initial tissue specimens (Huss et al., Prostate (2004);60:77-90). Human prostate primary xenografts were established as described previously and according to standard techniques (Huss et al., Prostate (2004);60:77-90). Briefly, to establish the xenografts, three-month-old male athymic nude mice (Hsd: athymic Nude/Nude; Harlan Sprague Dawley, Indianapolis, IN) that were to be hosts for prostate xenografts received subcutaneous implants of 12.5 mg of sustained release testosterone pellets (Innovative Research of America, Sarasota, Fla.) before transplantation of the prostate tissue to maintain serum testosterone levels in the host at about 4 ng/ml throughout the study, mimicking human serum levels. Human tissue designated as excess prostate was obtained from patients at the time of prostatectomy. Gross morphological assessment of the resected organ/tumor was the basis for identification of the specimens as originating in benign or tumor areas. An initial tissue specimen, at least 3 mm³, was cut from each tissue sample, placed in 10% formalin for fixation and paraffin embedded. Xenografts were established from the remaining tissue as described previously (Presnell, et al. (2001) Am. J. Pathol., 159: 855-860). Mice were observed weekly after implantation. One month post-implantation, the host mice were either euthanized, and the xenografts were harvested, placed in 10% formalin for fixation, and paraffin embedded, or the mice were castrated and the host euthanized and the xenografts harvested, fixed and prepared at various timepoints over the first seven days after castration or at 30 days post-castration. Paraffin blocks were sectioned (5 μm) onto ProbeOn Plus slides (Fisher Scientific International, Suwanee, Ga.).

In addition to the xenografts, Transgenic Adenocarcinoma of the Mouse Prostate (TRAMP) mice (Greenberg et al., (1995) PNAS 92:3439-43), a well accepted animal model for human prostate cancer, were used in this study. In this model, the PB-SV40 large T antigen transgene (SV40Tag) is expressed specifically in the epithelial cells of the murine prostate. Cells expressing the SV40Tag transgene can be detected by immunohistological methods using labeled monoclonal antibodies to SV40Tag. Adenocarcinoma of the prostate in TRAMP mice arises as early as eight weeks of age and is characterized by tumor cells that uniformly express high levels of AR and that regress upon castration. In contrast, poorly differentiated neuroendocrine-like carcinomas in TRAMP mice are characterized by expression of the neuroendocrine marker synaptophysin, and an absence, or weak and heterogeneous expression of AR. Neuroendocrine-like carcinomas are rare in young TRAMP mice, but arise rapidly in mice that are castrated between 12 and 14 weeks of age (Kaplan-Lefko et al., Prostate (2003); 55:219-37; Johnson et al., Prostate (2005); 62:322-38). Therefore, TRAMP mice are used herein to model the role of the prostate stem cell as the nidus of recurrent prostate cancer demonstrated by the neuroendocrine-like, poorly differentiated carcinomas that develop rapidly and aberrantly after castration in an androgen deprived prostate tissue microenvironment.

For the immunohistochemistry studies described herein, prostate tissue from surgical specimens, human prostate primary xenografts, and TRAMP mice was processed, and immunohistochemistry was performed as described previously and according to standard techniques (Huss et al., Prostate (2004); 60:77-90) and as further described herein.

For immunohistochemistry studies, tissue specimens were incubated with the following primary antibodies: polyclonal anti-AR (Upstate); rat monoclonal anti-BCRP (Bxp-53; Caltag Laboratories, Burlingame, Calif.; ref. 16); rabbit polyclonal antisynaptophysin (Zymed Laboratories, South San Francisco, Calif.); mouse monoclonal anti-BrdUrd (Sigma) (Example 4); rabbit polyclonal anti-Ki67 (Novocastra Laboratories, Newcastle upon Tyne, United Kingdom); mouse monoclonal anti-SV40Tag (BD PharMingen, San Diego, Calif.) (Example 4); mouse monoclonal anti-p63 (Santa Cruz, Santa Cruz, Calif.); mouse monoclonal 34βE12 anti-high molecular weight cytokeratin (Enzo Diagnostics, Farmingdale, N.Y.); goat polyclonal anti-Foxa2 (HNF3β; Santa Cruz) (Example 4); mouse monoclonal anti-smooth muscle α-actin (Sigma); or rabbit polyclonal anti-α-methylacyl-CoA racemase (AMACR/P504S; Biocare Medical, Walnut Creek, Calif.). Biotinylated secondary antibodies (Vector) were also utilized as indicated, and immunoreactive targets were detected using the Vectastain Elite ABC immunoperoxidase kit and 3,3V-diaminobenzidine, Nova Red (Vector), TrueBlue (KPL, Gaithersburg, Md.), or ABC Alkaline Phosphatase Kits I or III (Vector). Incubation without primary antibody and tissue specimens harvested from animals not injected with BrdUrd served as negative controls for immunohistochemical studies; mouse small intestine served as a positive control tissue for BrdUrd, Ki67, synaptophysin, p63, and BCRP immunohistochemical studies; prostate from intact TRAMP mouse served as the positive control tissue for SV40Tag and AR staining. In immunohistochemical analyses, areas/glands containing prostate cancer were identified by AMACR (racemase) staining of adjacent serial sections.

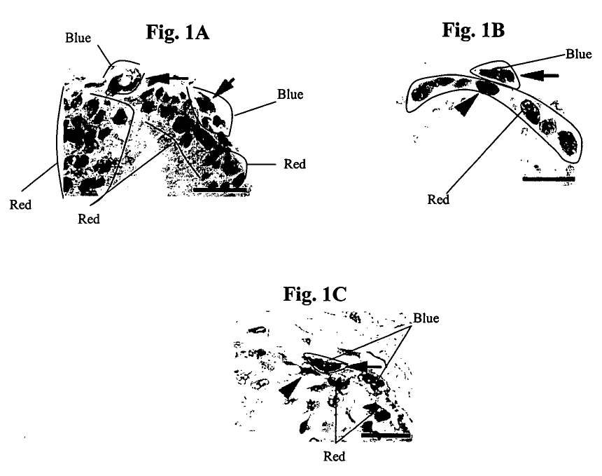

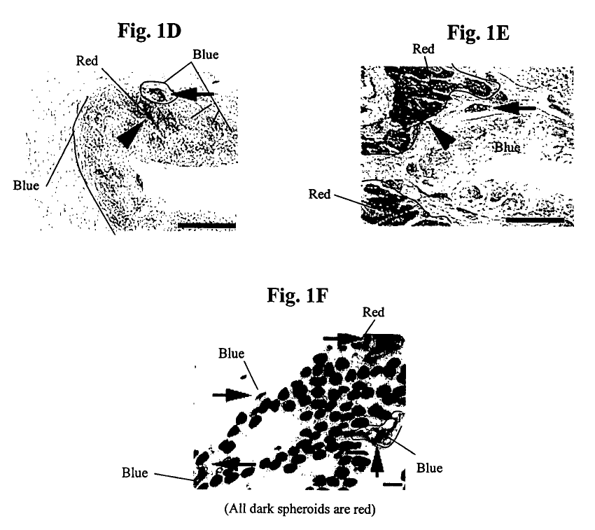

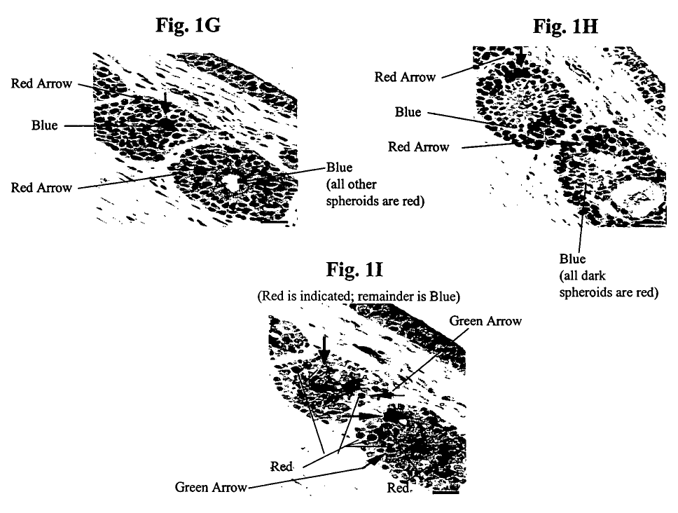

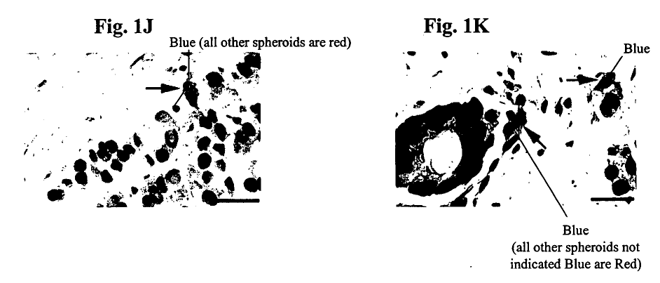

Prostate stem cells were identified in histologic sections of surgically resected human prostate tissue and determined to be rare, isolated cells that were BCRP+ and AR−; (FIG. 1A). Human prostate tissue was analyzed for BCRP+ cells that coexpressed the prostate basal cell markers p63 (FIG. 1B) and/or high molecular weight cytokeratin (FIG. 1C). Although the BCRP+ cells were contained within the p63/high molecular weight cytokeratin expressing basal cell compartment and were localized proximal to the basement membrane, BCRP+ cells did not co-express p63 or high molecular weight cytokeratin, indicating that prostate stem cells are not a subset of the basal cell population. In addition, BCRP+ cells did not co-express synaptophysin (FIG. 1D) or smooth muscle α-actin (FIG. 1E), demonstrating that prostate stem cells are neither neuroendocrine cells nor a component of the stromal compartment. Therefore, the minimal phenotype of human prostate stem cells is BCRP+, AR protein-negative (AR−), p63-negative (p63−), and synaptophysin negative (Syn−).

The percentage of BCRP+ cells (BCRP+/total epithelial cells) was determined based on a minimum of three x 200 microscopic fields, where at least 1,000 total epithelial cells were counted, and the percentage of BCRP+ cells was averaged for multiple patients or TRAMP mice. The percent of BCRP+ cells/gland was calculated by quantitation of the epithelial compartment from all identifiable glands per section, and means calculated for each patient or TRAMP mouse specimen. Means, SE, and one-way nonparametric ANOVA tests were done using Instat software (GraphPad, San Diego, Calif.). BCRP+/AR−/p63−/Syn− cells comprised 1.04% of the epithelial cells in benign human prostate glands, and were observed at a comparable frequency (0.57%) in glands containing prostate cancer (FIG. 1F; Table 1); prostate cancer was identified with AMACR in serial sections (AMACR staining not shown). In surgically resected human prostate tissue specimens, 35.7% of prostate glands contained at least a single BCRP+/AR−/p63−/Syn− cell. The primary xenografts established by subcutaneous transplantation of benign human prostate surgical specimens into androgen-supplemented immunocompromised hosts contained comparable proportions of glands with BCRP+ cells (24.4%) after 1 month of maintenance in an androgenic environment.

Thus, this Example demonstrates that human prostate stem cells are present in both benign and cancerous prostate glands and have a phenotype of BCRP+/AR−/p63−/Syn−. Furthermore, this Example demonstrates these cells are maintained in the primary xenografts demonstrating their ability to survive periods of androgen deprivation during transplantation.

### Example 2

This Example demonstrates the response of BCRP+ cells to androgen deprivation. In this regard, and as summarized in Table 1, the proportion of glands that contained BCRP+/AR−/p63−/Syn− cells were comparable in xenografts harvested from hosts after 1 month of establishment in an androgenic environment (24.4%), as well as xenografts harvested from hosts after 1 month of maintenance in an androgenic environment, castration, and maintenance for an additional 1 month postcastration in an androgen-deprived environment (29.5%). BCRP+/AR−/p63−/Syn− cells in human prostate histologic specimens were observed as isolated cells, with only rare glands (0.2%) containing multicellular foci of BCRP+/AR−/p63−/Syn− cells. In contrast, a large proportion of the glands (20.4%) in xenografts after 1 month of establishment in intact hosts contained foci of BCRP+ cells. Furthermore, castration of the host and maintenance of the xenografts under androgen deprived conditions increased the proportion of glands that contained foci of BCRP+/AR−/p63−/Syn− cells. In xenografts, during the initial 2 weeks postcastration, essentially all BCRP+/AR−/p63−/Syn− cells appeared to undergo focal expansion in response to androgen deprivation in that 32.2% of glands had foci of BCRP+ cells, a level comparable with the proportion of glands that contained isolated BCRP+ cells in the surgical specimen (P<0.005 comparing the three groups). The cells of the BCRP+ foci were AR− (FIG. 1G), p63− (FIG. 1H), and Syn− (data not shown), demonstrating that they were not secretory epithelial, basal epithelial, or neuroendocrine cells. Stimulation of the residual epithelial cell compartment in the involuted glands with exogenous androgen (for 48 hours) after 30 days of androgen deprivation induced focal proliferative activity in the epithelial compartment (Huss et al., Prostate (2004); 60:77-90) demonstrating the survival of a cell that could be induced to proliferate in the presence of androgen (a characteristic consistent with the phenotype of the prostate stem cell), and illustrating the potential efficacy of administering exogenous androgen in combination with a BCRP inhibitor. The proliferatively active cells (Ki67+) appeared to be immediate progeny of surviving BCRP+/AR−/p63−/Syn− cells because they were clustered adjacent to the BCRP+/AR−/p63−/Syn−/Ki67− cells (FIG. 1I) that survived castration. The loss of BCRP expression in the Ki67+ cells indicates entrance of the progeny of stem cells into the transit/amplifying compartment.

Thus, this Example demonstrates that BCRP+/AR−/p63−/Syn− cells in the prostates of prostate cancer patients and in primary xenografts of human prostate tissue maintained in castrate hosts can survive androgen deprivation and retain their proliferative potential in the absence of androgen. Therefore, BCRP+/AR−/p63−/Syn−cells are a potential nidus for recurrent cancer growth.

### Example 3

This Example demonstrates that, in a prostate cancer patient undergoing androgen deprivation therapy, prostate stem cells survive and may expand in response to androgen deprivation.

Because the prostate stem cell compartment in human prostate xenografts can survive androgen deprivation, and maintain proliferative capability, as demonstrated by a proliferative response to administration of exogenous androgen (Huss et al., Prostate (2004); 60:77-90), we also investigated whether prostate stem cells survive hormonal therapy in advanced prostate cancer patients. In a single patient for whom serial biopsy specimens after the initiation of androgen deprivation therapy were available, there was evidence of survival and possible expansion of the prostate stem cell compartment after 1 month of hormonal therapy (FIG. 1K). In contrast, BCRP+/AR− cells were observed as rare, isolated cells in a biopsy specimen harvested from this patient at the initiation of hormonal therapy (FIG. 1J).

Therefore, this Example demonstrates that, in an individual with prostate cancer, androgen deprivation does not kill prostate stem cells and may promote their expansion.

### Example 4

This Example demonstrates the presence of BCRP+ cells in recurrent tumors in castrate TRAMP mice.

The TRAMP mice used in this Example were transgenic F1 males (C57BL/6 TRAMP+/−x FVB; (reviewed in reference 14). Twelve-week-old TRAMP mice were implanted for two weeks with Alzet Minipumps (Durect Corp., Cupertino, Calif.) containing 200 μL of bromodeoxyuridine (BrdUrd; 60 mg/mL; Sigma) using standard techniques. The BrdUrd was administered in order to assess cellular proliferation using an anti BrdUrd antibody. Two days after pump removal, mice were castrated, or sham castrated, and prostates harvested at 0, 1, 2, 4, 7, and 14 days postcastration/sham castration (five mice per group).

In 14-week-old TRAMP mice that showed multiple well-established AR+ adenocarcinomas per gland. BCRP+/AR−/p63−/syn− cells represented 0.68% of the epithelial cells in the highly cellular prostate glands, with 38.6% of glands containing at least one BCRP+ cell (see Table 1, summarizing the percent of BCRP+/AR−/p63− cells and foci in surgical specimens of human benign prostate and prostate cancer, intact and postcastrate human prostate primary xenografts, and intact and postcastrate TRAMP). In TRAMP mice, the BCRP+ stem cells were localized to foci of AR− cells

TABLE 1

| | | Glands containing | Glands containing |
|---|---|---|---|
| | BCRP+/AR−/p63− cells/ | BCRP+/AR−/p63− cells/ | BCRP+/AR−/p63− foci/ |
| Prostate model | total epithelial cells | total glands | total glands |
| Human non-PCA | 1.04* ± 0.23 (n = 10) | 35.7 ± 5.6 (n = 10) | 0.2 ± 0.1^{H} (n = 10) |
| prostate surgical | | | |
| specimens | | | |
| Human PCA surgical | 0.57 ± 0.22 (n = 6) | ND | ND |
| specimens | | | |
| Human prostate | ND | 24.4 ± 7.6 (n = 7) | 20.4 ± 7.9^{H} (n = 7) |
| primary xenografts | | | |
| (30 d post | | | |
| transplantation) | | | |
| Postcastrate human | ND | 29.5 ± 9.7 (n = 11)^{I} | 32.2 ± 4.2^{H} (n = 34)^{'} |
| prostate primary | | | |
| xenografts | | | |
| Intact TRAMP | 0.68 ± 0.22 (n = 6) | 38.6 ± 7.6 (n = 6) | 2.0 ± 0.41 (n = 28) |
| Postcastrate TRAMP | ND | ND | 1.8 ± 0.4 (n = 23) |

Abbreviations:
PCA, prostate cancer;
n, sample number;
ND, not determined.
*Values are represented as a percentage.
^{H}P < 0.005 comparing human prostate non-prostate cancer surgical specimens, intact human prostate primary xenograft, and postcastrate human prostate primary xenografts.
^{I}Xenografts in hosts 30 days postcastration.
'Xenografts in hosts 1 to 14 days postcastration.

in glands of the ventral prostate, the site of origin of the greatest number of poorly differentiated carcinomas that arise after castration. Importantly, the frequency of BCRP+ cells was comparable in intact (2.0%) and castrated (1.8%) TRAMP mice (between 1 and 14 days postcastration), indicating the AR− foci that contained the BCRP+ stem cells were preexisting and not induced by androgen deprivation. However, cells in AR−/ SV40Tag+ foci in castrate mice are 9-fold (P<0.001) more likely to be proliferatively active (Ki67+) and express synaptophysin, compared with cells in the AR− foci in intact mice, indicating that androgen deprivation accelerates, and/or selects for, progression of the stem cell-driven foci that were refractory to androgen deprivation-induced involution.

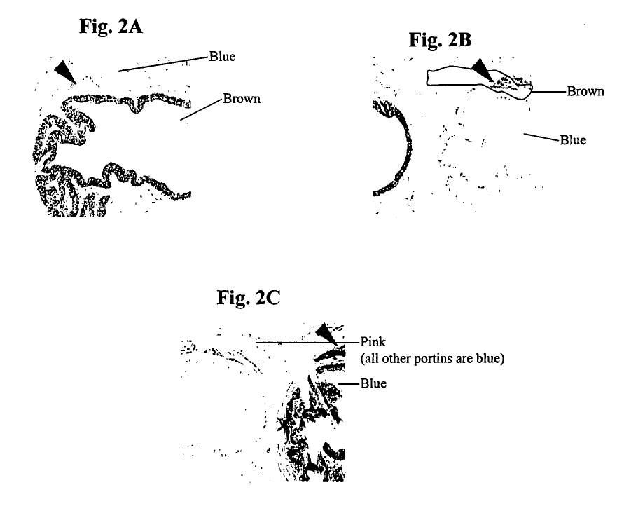

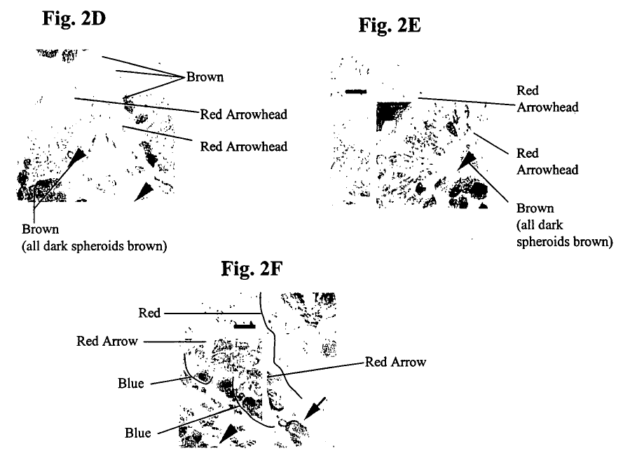

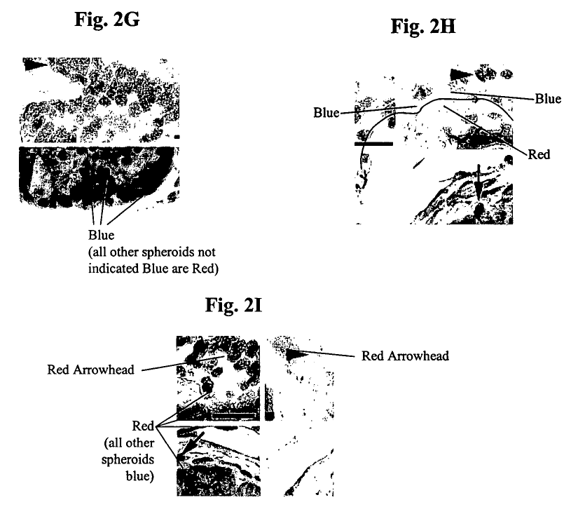

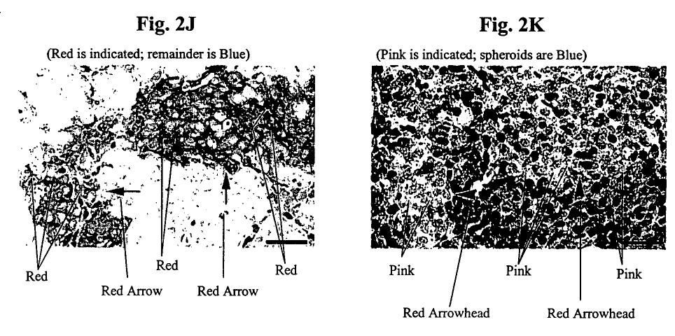

In castrate TRAMP mice, the cells in the AR− foci that contain prostate stem cells differ morphologically from the adenocarcinoma cells that express AR cytoplasmically (because the host is castrated and therefore lacks ligand) and that are SV40Tag−/Ki67− (FIG. 2A-C); (immunohistochemistry reagents used in this Example are detailed in Example 1). Expression of SV40Tag in the AR− foci in the androgen-deprived prostate is regulated potentially by the transcriptional regulatory protein Foxa2 (HNF3β; ref. 20), a member of the forkhead homeobox gene family (FIG. 2D). Foxa2 was expressed consistently in the AR−/SV40Tag+/Syn+/Ki67+ foci in prostates of both castrated (FIG. 2D-F) and intact TRAMP mice (data not shown). The BCRP+/AR− foci (FIG. 2F-J) arise rapidly postcastration (1 day; FIG. 2G) independent of AR-expressing, well-differentiated adenocarcinomas, possibly representing the nidus of the neuroendocrine-like carcinomas (FIG. 2J and K) that progress rapidly postcastration, but that also emerge eventually in intact TRAMP mice. That the BCRP+ cells represent stem cells in TRAMP mice is supported by the demonstration that BCRP+/AR− cells behave as label-retaining cells, a stem cell characteristic. BCRP+ cells that were prelabeled by incorporation of BrdUrd during a 2-week pulse before castration retained BrdUrd label for 2 weeks postcastration (FIG. 2H and I). In contrast, adenocarcinoma cells in the same prostates that also were prelabeled during the pulse period proliferated repeatedly during the chase period, diluting the incorporated BrdUrd to levels below detection.

The role of prostate stem cells as the nidus of poorly differentiated neuroendocrine-like carcinomas (recurrent cancer) was shown directly in a neuroendocrine-like carcinoma harvested from a castrated TRAMP animal that contained large focal areas of proliferatively active, BCRP+/AR−/Syn+ cells (FIG. 2J and K). The progression of neuroendocrine-like carcinomas in TRAMP mice, in contrast to the limited expansion of the stem cell foci in human xenografts of benign prostate, indicates the activation of a potent mechanism to replace androgen-mediated signaling in castrate TRAMP mice, possibly related to Foxa2− mediated activation of the SV40Tag transgene. Thus, this Example demonstrates that prostate stem cells serve as the nidus of recurrent prostate cancer in the TRAMP model, a well accepted model of human prostate cancer.

### Example 5

This Example demonstrates that BCRP mediates efflux of androgen from prostate stem cells.

Rat prostate progenitor (stem cell-like) cell lines (RPE, DP2, DP3, and DP4) were established from regenerating prostates of Fischer-344 rats and were maintained in prostate growth media (Presnell et al., Prostate Cancer Prostatic Dis (1999); 2:257-63) supplemented with 2% fetal bovine serum (FBS; Hyclone, Logan, UT) without androgen supplementation. For analysis of AR expression, RPE cells (5×10⁴ cells/chamber) were incubated in the presence or absence of 3 nmol/L dihydrotestosterone and/ or 50 μmol/L novobiocin (Sigma, St. Louis, Mo.) for 14 hours, fixed in 10% formalin for 5 minutes, and evaluated by immunocytochemistry as described previously (Presnell et al., Prostate Cancer Prostatic Dis (1999);2:257-63) utilizing polyclonal anti-AR (Upstate, Lake Placid, N.Y.) and biotinylated goat anti-rabbit antibody (Vector, Burlingame, Calif.). A subpopulation of RPE cells was selected for resistance to mitoxantrone (Mx-RPE) by continuous culture in increasing concentrations of mitoxantrone (0.2-4 μmol/L; Sigma).

To test whether BCRP-mediated efflux of androgen in prostate stem cells is the mechanism for maintenance of the prostate stem cell phenotype, the role of BCRP-mediated efflux of androgen (dihydrotestosterone) in maintenance of the phenotype of prostate stem cells was investigated indirectly using novobiocin, an inhibitor of BCRP-mediated efflux (Doyle et al., Oncogene (2003);22:7340-58; Shiozawa et al., Int. J. Cancer (2004); 108:146-51). In obtaining the data presented in this Example, RT-PCR amplification of BCRP mRNA and immunoblot analysis of AR and actin were performed as follows.

For RT-PCR analysis, total RNA was isolated with the RNeasy kit (Qiagen, Chatsworth, Calif.). Reverse transcription-PCR (RT-PCR) was done with the Advantage RT-PCR kit using primers specific for: G3PDH (BD Biosciences Clonetech, Palo Alto, Calif.), rat AR (17), and rat BCRP (forward primer: 5V-AGTCCGGAAAACAGCTGAGA-3V; (SEQ ID NO: 1) reverse primer: 5V-CCCATCACAACGTCATCTTG-3V). (SEQ ID NO: 2) PCR conditions consisted of 40 cycles of 1 minute of denaturation at 95° C., 1 minute of annealing at 56° C., and 90 seconds of primer extension at 72° C. PCR reactions containing RNA, but without the reverse transcription reaction, served as negative controls for each RT-PCR experiment. Rat ventral prostate, small intestine, and colon RNA were used as positive controls for each experiment.

For immunoblot analysis, RPE cells and rat tissues were homogenized on ice in lysis buffer [150 mmol/LNaCl, 1% Nonidet P-40, 0.5% Deoxycholic acid, 0.1% SDS, 50 mmol/L Tris-HCl (pH 8.0), 0.4 mmol/L EDTA (pH 8.0), 10% Glycerol] containing a cocktail of protease inhibitors (Complete Mini; Roche, Indianapolis, Ind.). Homogenates (50 μg of protein) were electrophoresed in 4% to 12% Bis-Tris gels (Invitrogen, Carlsbad, Calif.). Proteins were electroblotted to Hybond nitrocellulose membranes (Amersham Biosciences), and proteins of interest were immunodetected using primary antibodies for AR (Calbiochem, San Diego, Calif.), BCRP (Bxp-21; Chemicon, Temecula, Calif.), and actin (Santa Cruz). Secondary antibodies conjugated to horseradish peroxidase (Amersham Biosciences) were detected using an enhanced chemiluminescence detection system (Pierce, Rockford, Ill.). Rat small intestine and ventral prostate were included on each blot as positive controls for BCRP and AR expression. AR protein levels in immunoblots were evaluated and normalized to actin controls using ImageJ Software (O'Neill et al., Appl Theor Electrophor (1989); 1:163-7). Immunohistochemistry for AR expression and Hoechst 33342 fluorescence were analyzed using Optimas software (Media Cybernetics, Silver Springs, Md.).

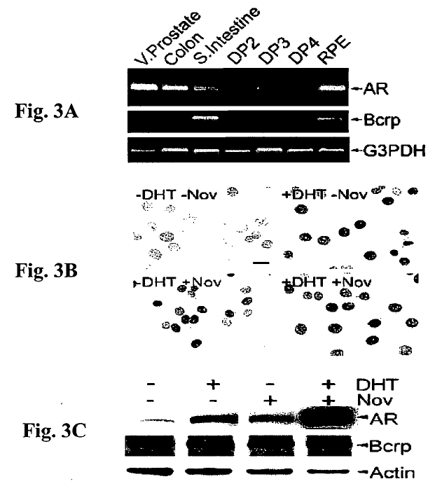

The rat prostate progenitor cell line RPE (Presnell et al., Prostate Cancer Prostatic Dis (1999); 2:257-63) expressed BCRP mRNA at levels comparable with rat small intestine and colon, and expressed AR mRNA at levels comparable with rat ventral prostate (FIG. 3A). However, RPE cells contained little detectable AR protein when cultured in 2.0% FBS (FIG. 3B and C), although they expressed substantial levels of AR mRNA. Consequently, the RPE cell line was utilized to examine the role of BCRP-mediated efflux of androgen in the regulation of the AR axis. Incubation of RPE cells with dihydrotestosterone, as a control, with novobiocin, or with novobiocin plus dihydrotestosterone, all resulted in the stabilization and nuclear translocation of AR protein as visualized by immunocytochemistry (FIG. 3B) (performed as previously described (Presnell et al., Prostate Cancer Prostatic Dis. (1999) Dec; 2(5/6): 257-263). Inhibition of BCRP-mediated transport by novobiocin alone resulted in a 2.4-fold increase in the level of nuclear-localized AR in RPE cells, compared with a 2.8-fold increase in response to incubation with dihydrotestosterone, and a 4.7-fold increase in response to incubation with dihydrotestosterone plus novobiocin (n>200 cells/group, P<0.0001). Immunoblot analysis of RPE cell lysates confirmed that novobiocin-mediated inhibition of BCRP function resulted in stabilization of intracellular AR protein, resulting in a 7-fold increase of AR protein in RPE cells incubated with novobiocin alone and a 25-fold increase of AR in cells incubated with novobiocin plus dihydrotestosterone (FIG. 3C). The more modest increase in AR stabilization measured by digital image analysis of immunocytochemistry compared with the much larger increase measured by immunoblot analysis reflects the limited dynamic range of the digital imaging technology, not a biologically significant difference between the experimental end points.

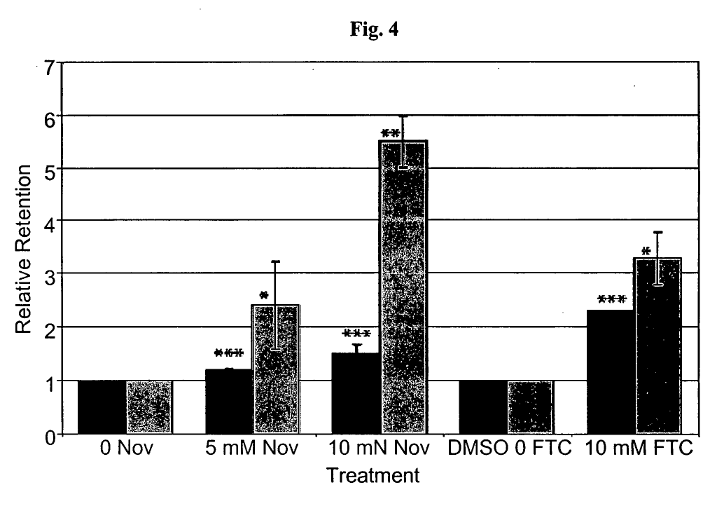

Additionally, novobiocin and fumitremorgin C were compared in RPE cells as inhibitors of BCRP-mediated efflux of Hoechst 33342, the marker utilized to identify the side population phenotype of stem cells. Fumitremorgin C is a specific inhibitor for BCRP-mediated transport. In contrast, whereas novobiocin inhibits BCRP selectively among the family of ABC cassette transporters, novobiocin is also a nonspecific inhibitor of cellular ATPases. Novobiocin was utilized in these studies in spite of the lack of specificity because fumitremorgin C is extremely neurotoxic, limiting use in vivo, whereas novobiocin is a widely utilized antibiotic with well tolerated toxicity in humans. Inhibition of BCRP-mediated transport in RPE cells by incubation with 5 or 10 mmol/L novobiocin resulted in a 1.2- and 1.45-fold increase in retention of Hoechst 33342, respectively, as determined by fluorescence digital image analysis (FIG. 4). Inhibition of BCRP-mediated transport with 10 μmol/L fumitremorgin C resulted in a 2.3-fold increase in nuclear Hoechst 33342 compared with the DMSO control (FIG. 4), confirming that BCRP is responsible for a majority of Hoechst efflux in RPE cells. The moderate increase in nuclear Hoechst 33342 dye in RPE cells with inhibited BCRP function reflects a combination of the limited dynamic range of digital imaging technology and the constitutive expression of MDR-1 in the RPE cells (data not shown) because MDR-1 also effectively transports Hoechst 33342 dye; however, novobiocin and fumitremorgin C inhibit only the BCRP-mediated component of Hoechst efflux, not MDR-1 function (Doyle et al., Oncogene (2003); 22:7340-58; Shiozawa et al., Int. J. Cancer (2004); 108:146-51). Therefore, BCRP-mediated efflux of androgen (dihydrotestosterone) was evaluated directly in a subline of RPE (Mx-RPE), which was selected by continuous culture in increasing levels of mitoxantrone, a prototypical substrate for BCRP transport (Doyle et al., Oncogene (2003); 22:7340-58). Mitoxantrone resistance in cell lines selected by the same protocol is usually associated with increased expression of BCRP due to gene amplification (Allen et al., Cancer Res; (2003); 63:1339-44). Thus, the Mx-RPE cells have a higher level of BCRP activity than RPE cells. Because of this, the difference in efflux between an inhibited Mx-RPE cell and a non-inhibited Mx-RPE cell is greater, and therefore easier to measure. Inhibition of the BCRP-mediated efflux of [3H]dihydrotestosterone in Mx-RPE cells by 5 or 10 mmol/L novobiocin or by 10 μmol/L fumitremorgin C resulted in 2.4-, 5.5-, and 3.3-fold increases in intracellular levels of [3H]dihydrotestosterone, respectively (FIG. 4), demonstrating that androgen is transported from Mx-RPE cells specifically by BCRP. In contrast to the RPE cells that express BCRP, three rat prostate epithelial progenitor cell lines also established in our laboratory (DP2, DP3, and DP4) do not express BCRP, and only DP3 expresses AR MRNA (FIG. 3A). Consequently, the DP3 cell line was utilized as a control to identify alternative mechanisms for intracellular accumulation of [3H]dihydrotestosterone in the absence of BCRP-mediated efflux, particularly mechanisms related to collateral effects of the ATPase inhibitory activity of novobiocin or to ligand-independent stabilization of AR protein. The DP3 cell line that lacked endogenous BCRP-mediated transport accumulated 3-fold higher concentrations of [3H]dihydrotestosterone than the Mx-RPE cells in the absence of novobiocin or fumitremorgin C. Novobiocin had no effect on the level of accumulation of intracellular [3H]dihydrotestosterone in the DP3 cell line, suggesting that BCRP, and not other ABC transporters or ATPase-dependent molecules, was the principal mechanism of cellular efflux of dihydrotestosterone.

Thus, this Example demonstrates that constitutive BCRP mediated efflux of androgen results in prostate progenitor cells that lack AR protein, despite expressing AR mRNA, and loss/inhibition of BCRP function allowed rapid cellular accumulation of dihydrotestosterone with stabilization and nuclear localization of AR protein.

### Example 6

This Example demonstrates that a prostate stem cell is the nidus of the poorly differentiated carcinomas that progress rapidly after castration in the TRAMP model, and that inhibition of BCRP activity in stem cells inhibits progression to poorly differentiated carcinomas.

Treatment of the TRAMP mice with novobiocin (25 mg/kg/5 times a week), or saline (physiologic saline/5 times a week), was initiated at 13 weeks of age and all mice were castrated at 14 weeks of age (1 week following initiation of treatment). TRAMP mice were treated until 19 weeks of age, at which time the prostates, lymph nodes, small intestine, and liver were harvested and processed for analysis using standard techniques. Paraffin embedded prostate tissue was sectioned at 5 μm until the entire prostate was sectioned (˜100-200 sections). Tissue sections spaced by 100 μm through the prostate were stained to detect expression of the SV40Tag transgene as described above.

The AR−/SV40Tag+ foci that contain the BCRP+/AR− stem cells are disclosed herein to be the nidus of the poorly differentiated carcinomas composed of SV40Tag+ cells that often co-expressed synaptophysin (a marker of prostatic neuroendocrine cells) and were AR−. As summarized in Table 2, five different patterns of expression of SV40Tag were identified in the prostates of the novobiocin treated and saline treated TRAMP mice five weeks after initiation of treatment. The five different patterns are: (a) foci of 1-5 positive cells within a differentiated prostate gland; (b) foci of 6-10 positive cells within a differentiated prostate gland; (c) foci of more then 10 positive cells within a differentiated prostate gland; (d) foci of more than 10 positive undifferentiated cells that represented small poorly differentiated carcinomas; and (e) large poorly differentiated carcinomas. The multiple focal growths observed within the prostate of individual animals demonstrated more than one of these patterns.

TABLE 2

| | #Foci/Animal | #Foci/Animal |
|---|---|---|
| Pattern of Foci of | in Novobiocin Tx | in Saline Tx |
| SV40Tag+ Cells | Animals | Animals |
| No Foci | 1/10 (0.1) | 1/14 (0.07) |
| 1 to 5 Cells | 6/10 (0.6) | 10/14 (0.7) |
| Differentiated | | |
| 6-10 cells | 11/10 (1.1) | 11/14 (0.8) |
| Differentiated | | |
| >10 cells | 13/10 (1.3)* | 4/14 (0.3) |
| Differentiated | | |
| >10 cells | 3/10 (0.3) | 13/14 (0.9)* |
| Small poorly | | |
| differentiated | | |
| carcinomas | | |
| Poorly | 3/10 (0.3) | 2/14 (0.1) |
| differentiated | | |
| carcinomas | | |

(*P < 0.0001, compared to other treatment group)

The number of small foci (<10 cells) of SV40Tag+ cells (both 1-5 cells and 5-10 cells) was similar in the novobiocin treated and control groups, indicating that there were comparable numbers of prostate stem cells present in the prostates of the two groups. In addition, there were comparable numbers of very large (palpable) tumors that arose rapidly in mice in both the control and treated group, suggesting the tumors were established and of substantial size before initiation of treatment. However, there were significant differences in the non-palpable foci between the novobiocin treated and control groups. There was a significant increase in the number of small (non-palpable) poorly differentiated carcinomas that were not apparent upon dissection in the control TRAMP mice compared to the novobiocin treated TRAMP mice. These tumors that were present in multiple serial sections most likely had derived from prostate stem cells during the interval between castration and harvest. In contrast, in the novobiocin treated TRAMP mice there was a significant increase in the number of small foci that contained more then 10 SV40Tag+ cells that demonstrated a differentiated phenotype compared to the control TRAMP mice. These small foci typically were not present through multiple serial sections.

Thus, in this Example, administration of novobiocin in TRAMP inhibited establishment of recurrent growth through inhibition of the expansion of the aberrant prostate stem cell compartment. The differentiated foci with more than 10 SV40Tag+ cells in the novobiocin treated TRAMP mice appear to represent the prostate stem cell, and its immediate progeny, that were forced to exit the stem cell compartment and differentiate in response to inhibition of BCRP function. In contrast, the large undifferentiated tumors that predominate in the saline control treated TRAMP animals indicate that the stem cell progressed to form the aggressive, undifferentiated tumors. Therefore, inhibition of BCRP mediated androgen efflux inhibits the progression from the prostate stem cell to a poorly differentiated carcinoma.

1. A method for reducing the aberrant growth of cells in a prostate tissue comprising administering to an individual in need of treatment a therapeutically effective amount of a composition comprising a BCRP inhibitor, wherein administration of the composition comprising the BCRP inhibitor reduces the aberrant growth of the cells in the prostate tissue.

2. The method of claim 1, wherein the individual has a prostate tumor.

3. The method of claim 1, wherein the individual is undergoing androgen deprivation.

4. The method of claim 1, wherein the BCRP inhibitor is selected from the group consisting of GF120918, fumitremorgin (FTC), FTC-type indolyl diketopiperazines, Ko143, antibodies to BCRP, CI 1033, Iressa (ZD1839), reserpine, VX-710, VX-853, estrogen agonists, antiestrogens, diethylstilbesterol, estrone, tamoxifen, toremifene, TAG-11, TAG-139 and novobiocin.

5. The method of claim 4, wherein the BCRP inhibitor is novobiocin.

6. The method of claim 1, wherein the composition comprising the BCRP inhibitor is administered concurrently or sequentially with a chemotherapeutic agent.

7. The method of claim 1, wherein the composition comprising the BCRP inhibitor is administered concurrently or sequentially with radiation therapy.

8. The method of claim 3, wherein the androgen deprivation therapy comprises administration of anti-androgenic hormones or castration.

9. The method of claim 1, wherein the BCRP inhibitor is administered concurrently or sequentially with an androgen.

10. The method of claim 2, wherein the prostate tumor is a recurrent prostate tumor.

11. The method of claim 1, wherein the cells in the prostate tissue are prostate stem cells.

12. The method of claim 1, wherein the individual has benign prostatic hyperplasia/hypertrophy (BPH).

13. The method of claim 13, wherein the BCRP inhibitor is administered concurrently or sequentially with an alpha-blocker, an alpha-reductase inhibitor, or saw palmetto, or combinations thereof.

14. A method for inhibiting the formation of foci in the prostate of an individual wherein the foci comprise aberrantly growing prostate cells, comprising administering to the individual a therapeutically effective amount of a composition comprising a BCRP inhibitor, wherein administration of the composition comprising the BCRP inhibitor inhibits formation of the foci.

15. The method of claim 14, wherein the individual has prostate cancer.

16. The method of claim 15, wherein the malignant cells are small, poorly differentiated carcinomas.

17. The method of claim 14, wherein the BCRP inhibitor is selected from the group consisting of GF120918, fumitremorgin (FTC), FTC-type indolyl diketopiperazines, Kol43, antibodies to BCRP, CI1033, Iressa (ZD1839), reserpine, VX-710, VX-853, estrogen agonists, antiestrogens, diethylstilbesterol, estrone, tamoxifen, toremifene, TAG-11, TAG-139 and novobiocin.

18. The method of claim 17, wherein the BCRP inhibitor is novobiocin.

19. The method of claim 14, wherein the individual is undergoing androgen deprivation therapy.

20. The method of claim 14, wherein the individual has BPH.
